(19) United States
(12) Patent Application Publication
(10) Pub. No.: US 2014/0221908 A1
(43) Pub. Date: Aug. 7, 2014

(54) SURFACTANT PORTED DEVICE FOR TREATMENT OF BLEPHARITIS AND APPLICATIONS OF SAME

(71) Applicants: Jeffrey Sonsino, Nashville, TN (US); John P. Wikswo, Brentwood, TN (US); David Schaffer, Nashville, TN (US); Kevin Seale, Nashville, TN (US); Phillip C. Samson, Nashville, TN (US); Ronald S. Reiserer, Nashville, TN (US)

(72) Inventors: Jeffrey Sonsino, Nashville, TN (US); John P. Wikswo, Brentwood, TN (US); David Schaffer, Nashville, TN (US); Kevin Seale, Nashville, TN (US); Phillip C. Samson, Nashville, TN (US); Ronald S. Reiserer, Nashville, TN (US)

(73) Assignee: VANDERBILT UNIVERSITY, Nashville, TN (US)

(21) Appl. No.: 14/129,755

(22) PCT Filed: Jul. 2, 2012
(86) PCT No.: PCT/US2012/045298

§ 371 Date: Apr. 7, 2014

Related U.S. Application Data
(60) Provisional application No. 61/504,092, filed on Jul. 1, 2011.

(51) Int. Cl. A61F 9/007

(52) U.S. Cl. CPC A61F 9/00772
USPC 604/28; 604/35; 604/294

(57) ABSTRACT

Certain aspects of the present disclosure are directed to a system for treating a gland of a patient. The system includes a handheld device including a probe, a tip of the probe being formed with a suction opening and a delivery opening; a backend apparatus including a fluid reservoir configured to contain a fluid, an agent reservoir configured to contain agent particles, and a suction force generator; a delivery channel in fluid communication with the delivery opening and the fluid reservoir and the agent reservoir, the delivery channel allowing a mixture of the fluid and the agent particles to travel through the delivery channel and exit at the delivery opening; and a suction channel in fluid communication with the suction opening and the suction force generator, the suction channel allowing a suction force being generated at the suction opening by the suction force generator.

### CROSS-REFERENCE TO RELATED PATENT APPLICATION

This PCT application claims the benefit of U.S. Provisional Application No. 61/504,092, filed on Jul. 1, 2011 and entitled “Surfactant Ported Device For Treatment of Blepharitis and Applications of Same.” The disclosure of the above application is incorporated herein by reference in its entirety.

Some references, which may include patents, patent applications and various publications, are cited in a reference list and discussed in the description of this disclosure. The citation and/or discussion of such references is provided merely to clarify the description of the present disclosure and is not an admission that any such reference is “prior art” to the disclosure described herein. All references listed, cited and/or discussed in this specification are incorporated herein by reference in their entireties and to the same extent as if each reference was individually incorporated by reference. In terms of notation, hereinafter, bracketed “n” represents the n^{th} reference cited in the reference list. For example, [1] represents the 1^{st} reference cited in the reference list, namely, Lemp M A, Nichols K K. Blepharitis in the United States 2009: a survey-based perspective on prevalence and treatment. Ocul Surf. 2009 April; 7(2 Suppl):S1-S14.

### FIELD

The present disclosure relates generally to apparatus and method of treating glands of a patient and, in particular, of treating meibomian glands in eyelids of a patient.

### BACKGROUND

The human body contains a number of glands including the lacrimal and meibomian glands of the eye, the sebaceous or pilo-sebaceous hair glands on the face and underarms, and the mammary glands in the breasts. These glands may malfunction due to age, irritation, environmental conditions, cellular debris, inflammation, hormonal imbalance and other causes. One common malfunction is the restriction or stoppage of the natural flow of secretions out of the gland.

While sometimes the description that follows is directed to the meibomian glands of the eye, it will be understood that description may also apply to other external glands of the body. With particular reference to the human eye, the tear film covering the ocular surfaces is composed of three layers. The innermost layer in contact with the ocular surface is the mucus layer. The middle layer comprising the bulk of the tear film is the aqueous layer, and the outermost layer is a thin (less than 250 nm) layer comprised of many lipids known as “meibum” or “sebum.” The sebum is secreted by the meibomian glands, enlarged specialized sebaceous-type glands located on both the upper and lower eyelids, with orifices designed to discharge the lipid secretions onto the lid margins, thus forming the lipid layer of the tear film. The typical upper lid has approximately 25 meibomian glands and the lower lid has approximately 20 meibomian glands, which are somewhat larger than those located in the upper lid. The meibomian gland comprises various sac-like acini which discharge the secretion into the duct of the gland. The secretion then passes into the orifices which are surrounded by smooth muscle tissue and the muscle of Riolan which are presumed to aid in the expression of sebum. The meibomian gland orifices open on the lid margin usually along the mucocutaneous junction also known as the gray line. The meibomian gland orifices are assumed to open with blinking and release minute amounts of sebum secretions onto the lid margin and then into the inferior tear meniscus. The lipid “sebum” in the tear meniscus is spread upward and over the tear film of the open eye by the upward blink action. If the lipid secretions are optimal, and adequate lipid layer is maintained at the air interface to minimize evaporation and prevent dry eye states. If the lipid secretions are inadequate the lipid layer is not adequate to minimize evaporation with resulting rapid evaporation leading to dry eye states. Thus, it will be seen that a defective lipid layer or an incorrect quantity or quality of such lipids can result in accelerated evaporation of the aqueous layer which, in turn, causes symptoms which may include symptoms such as dryness, scratching, irritation, burning, tearing, redness, and itchiness, which are collectively be referred to as “dry eye” symptoms.

Dry eye states have many etiologies. A common cause of common dry eye states is the condition known as “meibomian gland dysfunction”, a disorder where the glands are obstructed or occluded. As employed herein the terms “occluded” and “obstruction” as they relate to meibomian gland dysfunction are defined as partially or completely blocked or plugged meibomian glands. If completely obstructed the gland cannot secrete. If partially or intermittently occluded the gland may secrete either normal or decreased amounts of sebum. More usually the secretions are altered having semi-solid, thickened, congested secretions, frequently described as inspissated. The secretions may be clear or yellowish, the latter indicating possible infection. Meibomitis, an inflammation of the meibomian glands leading to their dysfunction, is usually accompanied by blepharitis (inflammation of the lids). Meibomian gland dysfunction may accompany meibomitis, or meibomian gland dysfunction may be present without obvious lid inflammation. Meibomian gland dysfunction is frequently the result of keratotic obstruction of the individual meibomian gland orifices and/or the central duct (canal) of the gland which compromises the secretory functions of the individual meibomian glands. More particularly, these keratotic obstructions include a combination of desquamated epithelial cells, keratin, sebaceous ground substance, and bacteria. While meibomitis is obvious by inspection of the external lids, meibomian gland dysfunction may not be obvious even when examined with the magnification of the slit-lamp biomicroscope, since there may not be external signs or the external signs may be so minimal that they are overlooked. The external signs of meibomian gland dysfunction may be limited to subtle alterations of the meibomian gland orifices, subtle or pronounced overgrowth of epithelium over the orifices, and pouting of the orifices of the glands with congealed material acting as the obstructive material under the epithelia overgrowth resulting in the pouting of the orifices.

When the flow of secretions from the meibomian gland is restricted due to the existence of an obstruction, epithelial cells on the eyelid margin tend to grow over the gland orifice thus further restricting sebum flow and exacerbating the dry eye condition.

Blepharitis, or inflammation of eyelids, is a significant problem facing about ⅓ of the population over 50 years old. [1] The source of inflammation is blockage of the meibomian glands which line the eyelids due to debris including meibum, sebum, lipids and protein from the tears, and dead and devitalized epithelial cells (from the meibomian gland itself). The meibomian gland becomes blocked but continues to secrete meibum. The result in severe cases are styes (or internal hordeolae). [2] Bacteria which are normally found within the meibomian gland overpopulate due to the increased food supply. A lower grade, but much more prevalent problem associated with blocked meibomian glands is a persistent and nagging dry eye.

Typically blepharitis is treated and then managed and will ebb and flow according to exogenous and endogenous factors. The condition is currently treated by having patients apply hot compresses to the eyelids, lid massage, and application of surfactants to the eyelid. Patient compliance with this regimen is notoriously low.

Oral or topical antibiotics are given to reduce the bacterial load in the gland, but are associated with significant side effects, such as gastro-intestinal distress, inability to tolerate sunlight, and allergic reactions among other complications.

Topical anti-inflammatories are given to reduce eyelid inflammation, but do not penetrate deep into the gland.

A physician in Argentina is currently treating blepharitis by using a micro needle to push the debris cap over the meibomian gland into the gland itself. This questionably barbaric attempt to clear the gland opening is not optimal for obvious reasons.

The title of a recent article in the Harvard Woman's Health Watch sums up the efficacy of current treatment and practitioner frustration in managing the condition: “By the way, doctor. I suffer from blepharitis and have tried many medications, but the condition always returns. How can I cure it?”[3]

Therefore, a heretofore unaddressed need exists in the art to address the aforementioned deficiencies and inadequacies.

### SUMMARY

Certain aspects of the present disclosure are directed to a system for treating a gland of a patient. The system includes a handheld device including a probe, a tip of the probe being formed with a suction opening and a delivery opening; a backend apparatus including a fluid reservoir configured to contain a fluid, an agent reservoir configured to contain agent particles, and a suction force generator; a delivery channel in fluid communication with the delivery opening and the fluid reservoir and the agent reservoir, the delivery channel allowing a mixture of the fluid and the agent particles to travel through the delivery channel and exit at the delivery opening; and a suction channel in fluid communication with the suction opening and the suction force generator, the suction channel allowing a suction force being generated at the suction opening by the suction force generator.

In certain embodiments, the tip further includes a shoe adapted to be placed over a gland of a patient, a first side of the shoe having at least one suction area including the suction opening and at least one delivery area including the delivery opening, and the suction area surrounding the delivery area.

In certain embodiments, the delivering area has a plurality of discrete areas, and the suction area has a plurality of discrete areas.

In certain embodiments, the shoe has a plurality of protrusions placed adjacent to, and protruding away from, the suction area.

In certain embodiments, the handheld device further comprises an agitator at the tip of the probe and configured to generate an agitating force.

In certain embodiments, the backend apparatus has a delivery module in fluid communication with the fluid reservoir and the agent reservoir, and the delivery module is configured to utilize the fluid to deliver the agent particles to the delivery opening.

In certain embodiments, the gland is a meibomian gland in an eyelid of a patient, and the probe includes a heater at the tip and configured to heat the fluid exiting at the delivery opening to a predetermined temperature such that the heated fluid is sufficient to liquefy or soften an obstruction within or at the meibomian gland.

In certain embodiments, the backend apparatus has a heating source configured to heat the fluid in the fluid reservoir to generate a heated fluid. In certain embodiments, the agent particles include nanospheres each containing a medicine. In certain embodiments, a first portion of the nanospheres each include a first medicine, and a second portion of the nanospheres each include a second medicine. In certain embodiments, the nanospheres each include a drug cocktail containing at least one of antibiotic, anti-inflammatory, vasodilator or vasoconstrictor, anti-keritin, anti Veg-F, and salicylic acid.

Certain aspects of the present disclosure are directed to a method for treating a meibomian gland of a patient, The method including: placing a tip of a probe of a handheld device over a meibomian gland on an eyelid, the tip having a suction opening and a delivery opening; and operating a backend apparatus, the backend apparatus including a fluid reservoir having a fluid, an agent reservoir having agent particles, and a suction force generator, such that a delivery channel delivers a mixture of the fluid and the agent particles to exit at the delivery opening and to arrive at the meibomian gland, the delivery channel being in fluid communication with the delivery opening and the fluid reservoir and the agent reservoir; and such that a suction force is generated at the suction opening by the suction force generator and draws a material at the meibomian gland into a suction channel through the suction opening, the suction channel being in fluid communication with the suction opening and the suction force generator.

Certain embodiments of the present disclosure are directed to a handheld device for treating a target region of a patient. The handheld device includes: an elongate housing; a recess formed at a tip of the elongate housing; a head cartridge placed in the recess, including a delivery manifold having a plurality of delivery ports at a treatment side of the delivery manifold, the delivery manifold being in fluid communication with a fluid reservoir through a main port of, and at a backend side of, the delivery manifold, the delivery manifold allowing a fluid contained in the fluid reservoir to exit at the delivery ports; and a contact surface adjacent to the recess, the contact surface having a rugged texture surface.

These and other aspects of the present disclosure will become apparent from the following description of the preferred embodiment taken in conjunction with the following drawings and their captions, although variations and modifications therein may be affected without departing from the spirit and scope of the novel concepts of the disclosure.

### BRIEF DESCRIPTION OF THE DRAWINGS

The drawings described below are for illustration purposes only. The drawings are not intended to limit the scope of the present teachings in any way.

FIG. 1 illustrates a handheld device and a backend apparatus in accordance with certain embodiments of the present disclosure;

FIG. 2 illustrates a shoe at a probe tip of a handheld device in accordance with certain embodiments of the present disclosure;

FIG. 3 illustrates delivery tubes and suction tubes at the probe tip in accordance with certain embodiments of the present disclosure;

FIGS. 4(A)-(B) illustrate configurations of delivery tubes and suction tubes at the probe tip in accordance with certain embodiments of the present disclosure;

FIGS. 5(A)-(B) illustrate a manifold in accordance with certain embodiments of the present disclosure;

FIGS. 6(A)-(D) illustrate a handheld device in accordance with certain embodiments in use for treating a meibomian gland

FIG. 7 illustrates another handheld device in accordance with certain embodiments of the present disclosure;

FIG. 8 illustrates the tip of the handheld device in accordance with certain embodiments of the present disclosure; and

FIGS. 9(A)-(C) illustrate a head cartridge in accordance with certain embodiments of the present disclosure.

### DETAILED DESCRIPTION

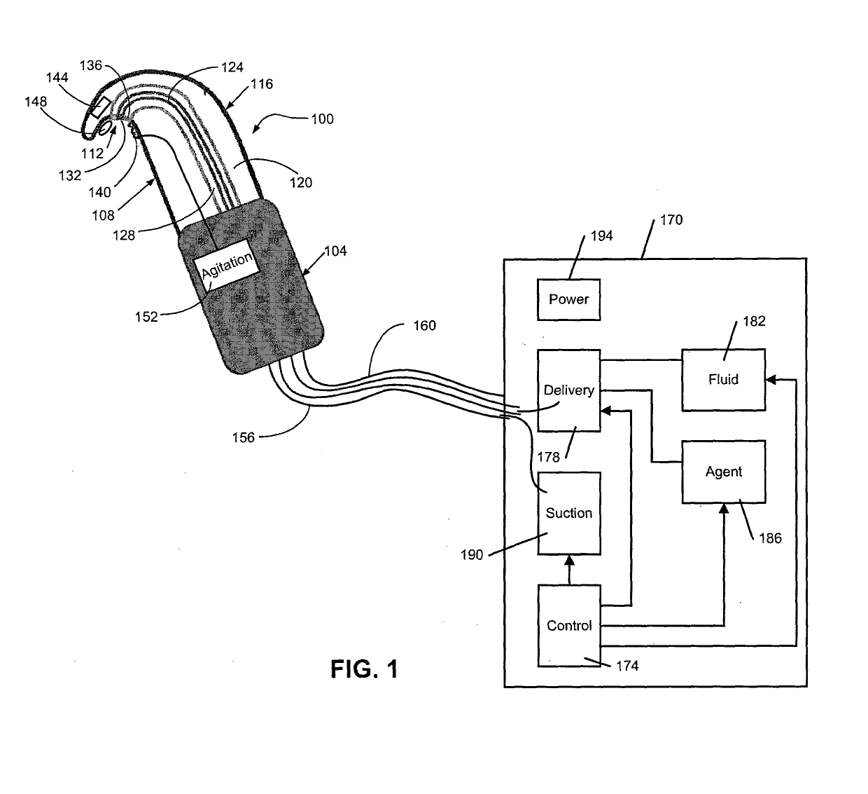

The present disclosure is more particularly described in the following examples that are intended as illustrative only since numerous modifications and variations therein will be apparent to those skilled in the art. Various embodiments of the disclosure are now described in detail. Referring to the drawings, FIGS. 1-6, like numerals, if any, indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present disclosure. Additionally, some terms used in this specification are more specifically defined below.

The terms used in this specification generally have their ordinary meanings in the art, within the context of the disclosure, and in the specific context where each term is used. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the disclosure or of any exemplified term. Likewise, the disclosure is not limited to various embodiments given in this specification.

Unless otherwise defined, all technical and scientific terms used herein have the same meaning as commonly understood by one of ordinary skill in the art to which this disclosure pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, “nanoscopic-scale,” “nanoscopic,” “nanometer-scale,” “nanoscale,” “nanocomposites,” “nanoparticles,” the “nano-” prefix, and the like generally refers to elements or articles having widths or diameters of less than about 1 μm, preferably less than about 100 nm in some cases. In all embodiments, specified widths can be smallest width (i.e. a width as specified where, at that location, the article can have a larger width in a different dimension), or largest width (i.e. where, at that location, the article's width is no wider than as specified, but can have a length that is greater).

As used herein, “plurality” means two or more.

As used herein, the terms “comprising,” “including,” “carrying,” “having,” “containing,” “involving,” and the like are to be understood to be open-ended, i.e., to mean including but not limited to.

As used herein, terms such as “first”, “second”, “third”, and the like are used for distinguishing various elements, members, regions, layers, and areas from others. Therefore, the terms such as “first”, “second”, “third”, and the like do not limit the number of the elements, members, regions, layers, areas, or the like. Further, for example, the term “first” can be replaced with the term “second”, “third”, or the like.

As used herein, terms for describing spatial arrangement, such as “over”, “above”, “under”, “below”, “laterally”, “right”, “left”, “obliquely”, “behind”, “front”, “inside”, “outside”, and “in” are often used for briefly showing a relationship between an element and another element or between a feature and another feature with reference to a diagram. Note that embodiments of the present disclosure are not limited to this, and such terms for describing spatial arrangement can indicate not only the direction illustrated in a diagram but also another direction. For example, when it is explicitly described that “B is over A”, it does not necessarily mean that B is placed over A, and can include the case where B is placed under A because a device in a diagram can be inverted or rotated by 180°. Accordingly, “over” can refer to the direction described by “under” in addition to the direction described by “over”. Note that embodiments of the present disclosure are not limited to this, and “over” can refer to any of the other directions described by “laterally”, “right”, “left”, “obliquely”, “behind”, “front”, “inside”, “outside”, and “in” in addition to the directions described by “over” and “under” because the device in the diagram can be rotated in a variety of directions. That is, the terms for describing spatial arrangement can be construed adequately depending on the situation.

FIG. 1 illustrates a handheld device 100 in accordance with certain embodiments of the present disclosure. The handheld device 100 can be used for treating a target such as a gland, an organ, a tissue, or a wound, in particular an external one, of a patient. The device 100 has a handle 104 and a probe 108. The probe 108 has a probe tip 112 and a probe body 116. The probe tip 112 can be sized to suit a particular gland, organ, tissue, or wound. Further, in order to treat a meibomian gland, the probe tip 112 can have a curvature, such as a hooked shape, which allows easy manipulation by a user to place the probe tip 112 behind the eyelid and to ergonomically access the meibomian gland.

In certain embodiments, the probe body 116 has a chamber 120 that includes a delivery channel 124 and a suction channel 128. The delivery channel 124 can deliver fluids such as vapor, steam, warm mist humidified air, lipid emulsifier, medicine/drugs, gas, solution, liquid gas, surfactant, chemical/pharmacological agents, medicine/drugs, nanospheres containing medicines, etc. to the target, The suction channel 128 can transfer a suction force to the target, and thus can extract obstructions, cellular tissues, and materials that were supplied by the delivery channel 124 and that now have been used. The delivery channel 124 and the suction channel 128 are exposed at the probe tip 112 and define one or more delivery openings 132 and one or more suction openings 136 of the probe tip 112, respectively. In certain embodiments, the suction channel 128 surrounds the delivery channel 124. The probe tip 112 can have central delivery areas having the delivery openings 132 that supply fluid used to treat the target, as well as suction areas, having the suction openings 136 and around the delivery areas, that extract the obstructions, tissues, and other materials.

An agitator 140, such as a brush or a fluid jet device, is placed at the probe tip 112 and can be used to disrupt the target. The agitator 140 can apply agitating forces, such as vibratory type forces, including those generated mechanically or those using fluid type devices or mechanisms. For example, the agitator 140 can vibrate at a frequency in a predetermined range, for example, from about 1,000 Hz to about 20,000 Hz. The agitator 140 can vibrate at a varied frequency in accordance with a predetermined pattern or routine. For example, the agitator 140 can start at a low frequency and finish at a high frequency. In certain embodiments, the agitator 140, such as a fluid jet, can eject fluids at a pressure in a predetermined range in accordance with a predetermined pulse train.

In certain embodiments, the probe tip 112 can have a heater 144 placed adjacent to the delivery area. The heater 144 provides thermal energy, i.e., heats, to the fluids in the delivery channel 124 and can be used to control the temperature of the fluids exiting the delivery channel 124 at the delivery openings 132. The temperature can be controlled in a predetermined range. The probe tip 112 can also have a temperature sensor 148 adjacent to the delivery area that measures the temperature of the exiting fluids and to provide feedback to the heater 144 or a control module controlling the heater 144. The heater 144 can be turned on or off based on the feedback provided by the temperature sensor 148.

The handle 104 supports the probe 108. The delivery channel 124 and the suction channel 128 are placed through the handle 104. For example, the delivery channel 124 and the suction channel 128 can be implemented by tubes having diameters in a predetermined range. The handle 104 can also include an agitation module 152 that supplies the agitating force mechanically or through fluids to the agitator 140.

In certain embodiments, the delivery channel 124 is further implemented by a delivery tubing 156 connecting the device with a backend apparatus 170; the suction channel 128 is further implements by a suction tubing 160 connecting the device 100 with the backend apparatus 170. The backend apparatus 170 includes a control module 174, a delivery module 178, a fluid module 182, an agent module 186, a suction module 190, and a power module 194. As will be discussed in more detail later, the fluid module 182 can release a fluid into the delivery module 178. The agent module 186 can release agent particles into the delivery module 178. The delivery module 178 can release the fluid, such as a vapor, and agent particles, such as nanospheres containing medications, through the delivery channel 124, 156 to the probe tip 112. The fluid module 182 can have a fluid reservoir having a fluid. The agent module 186 can have an agent reservoir having agent particles. The suction module 190 can have a suction force generator such as a suction pump that generates a vacuum, which through the suction channel 128 cause a suction force at the probe tip 112. The control module 174 allows a practitioner to control the release of the fluid and/or agent particles to exit at the probe tip 112 as well as to control the suction force generated at the probe tip 112. The power module 194 can receive power supply from a power source and energize the other modules in the backend apparatus 170. In certain embodiments, the power module 194 may include a power source which may be direct current (battery powered) or alternating current (wall socket) as desired.

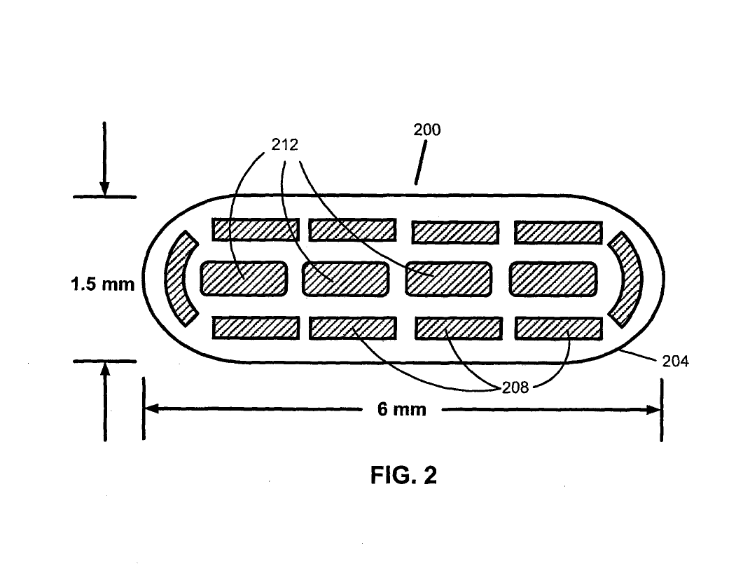

In certain embodiments, the probe tip 112 has a shoe 200 that is to be placed over the target such as an external gland. FIG. 2 illustrates an example of such a shoe 200. A front side 204 (or the treatment side) of the shoe 200 has suction areas 208 and delivery areas. The front side 204 can have a rounded rectangular shape. For example, the front side 204 can have a length of about 6 mm and a width of about 1.5 mm. The delivery area 212 can be at the inner portion of (e.g., in the middle) of the front side 204. The suction area can be at the outer portion of the front side 204. In certain embodiments, the suction area can surround the delivery area 212. One or more delivery passages are crossing through the shoe 200 between the front side 204 and the back side (or the backend side) of the shoe 200. One end of the delivery passages terminate at the delivery area 212. One or more delivery tubes 304 implementing the delivery channel 124 can be placed through the delivery passages. Likewise, one or more suction passages are crossing through the shoe 200 between the front side 204 and the back side of the shoe 200. One end of the suction passages terminate at the suction area. One or more suction tubes implementing the suction channel 128 can be placed through the suction passages.

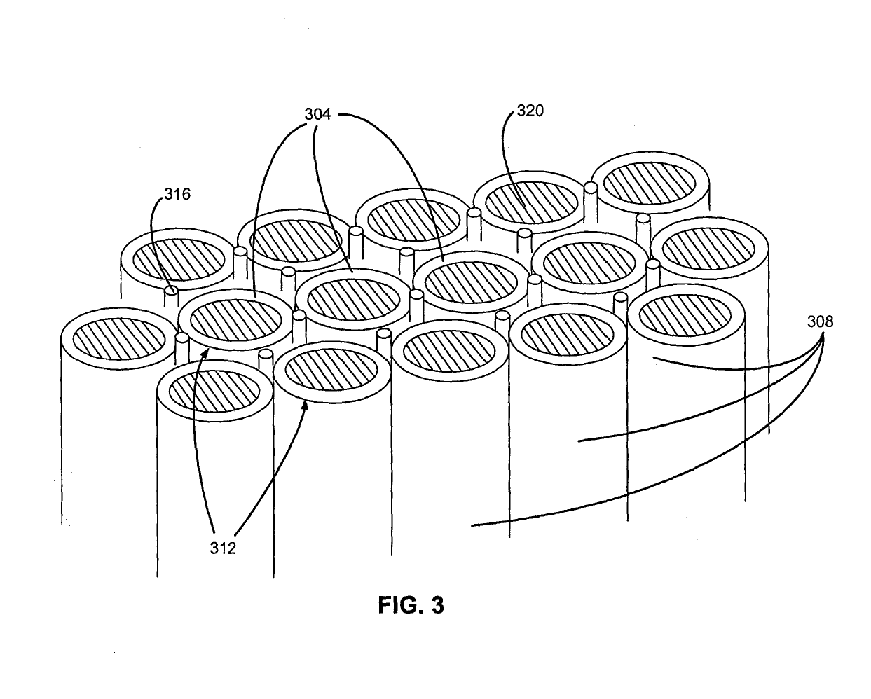

FIG. 3 illustrates that, in certain embodiments, the delivery tubes 304 and the suction tubes 308 can have an elongated cylindrical shape. They can be made of silicone or other similar materials. In certain embodiments, the front portions of the delivery tubes 304 and the suction tubes 308 can be embedded in the delivery passages such that the front side 204 of the shoe 200 defines an outer boundary of the probe tip 112. In certain embodiments, the front portions of the delivery tubes 304 and the suction tubes 308 can pass through the delivery passages and extend out from the delivery passages, such that the front tips 312 of the delivery tubes 304 and the suction tubes 308 define the outer boundary of the probe tip 112.

FIG. 3 further illustrates that, in certain embodiments, the probe tip 112, for example the shoe 200, can have a plurality of protrusions 316 placed adjacent to, and protruding away from, the suction area. For example, the protrusions 316 can be standoff pegs configured to separate the tissue of the eyelid from the suction opening 320 when the probe tip 112 is placed over a meibomian gland. In this example, the front tips 312 of the standoff pegs are protruding away from the suction opening 320 defined by the tips of the suction tubes 308. Therefore, when the probe tip 112 is placed over the gland, the standoff pegs 316 cause the tissues of the gland or eyelid at and adjacent to the standoff pegs 316 to be separated from the tips of the suction tubes 308 and not to be in close contact with suction tubes 308.

In certain embodiments, the standoff pegs 316 can also function as the agitator that provides mechanical forces. For example, the standoff pegs 316 can be vibrated by mechanism as known by one skilled in the art at a frequency in a predetermined range.

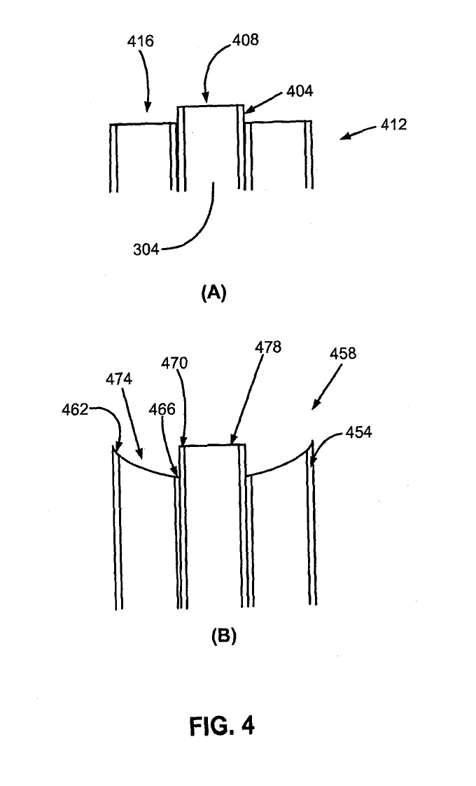

FIG. 4(A) illustrates that, in certain embodiments, the tip 404 of a delivery tube 304, defining the delivery opening 408, is extending away from the tips 412 of the suction tubes 308, defining the suction openings 416, placed adjacent to the delivery tube 304. Therefore, when the probe tip 112 is placed over a target, the tips 404 of the delivery tube 304 causes the tissues at and adjacent to the target to be separated from the tips 412 of suction tubes 308 and not to be in close contact with suction tubes 308.

FIG. 4(B) shows that, in certain embodiments, the tip 454 of the suction tube 308 defines a slanted plane. In other words, the slanted suction opening 458 defined by the tip 454 has a high point 462 and a low point 466 along the axis of the suction tube 308. A delivery tube 304 can be placed adjacent to such a suction tube 308. A side surface 470 of the delivery tube 304 and the tip 454 of the suction tube 308 define a space 474 between the slanted plane defined by the suction tube 308 and a plane defined by the tip 478 of the delivery tube 304. Therefore, when the probe tip 112 is placed over the target, that defined space 474 causes the tissues at and adjacent to the target to be separated from the tips 454 of suction tube 308 and not to be in close contact with suction tube 308.

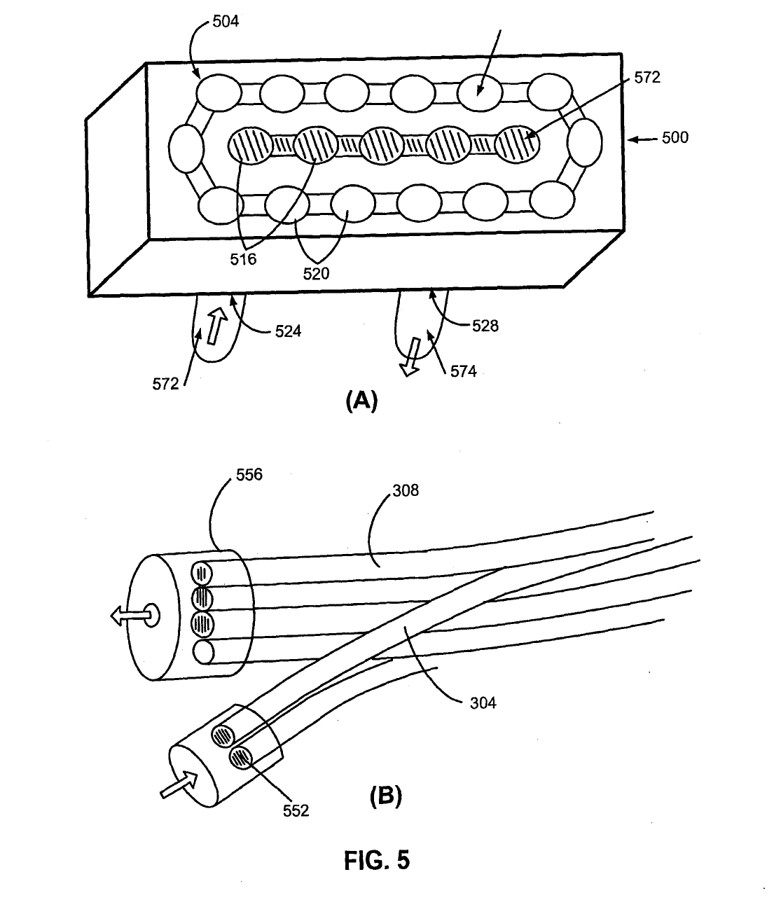

In certain embodiments, the delivery tubes 304 and the suction tubes 308, extending from the backside (backend side) of the shoe 200, are coupled with a backend manifold. FIG. 5(A) illustrates an exemplary manifold 500. The front side 504 of the manifold 500 has suction areas 508 and delivery areas 512. The delivery areas 512 can have one or more front delivery openings 516, which are defined by the delivery passages crossing through the manifold, at the front side of the manifold. The delivery tubes 304 pass through the front delivery openings 512 and enter the delivery passages at the front side 504 of the manifold 500. The delivery passages also define one or more back delivery openings 524 at the backside of the manifold 500. In certain embodiments, the delivery passage has multiple branches at the front end 504 of the manifold 500. Each of the delivery tubes 304 enters into a respective branch of the delivery passage. The multiple branches converge into a single branch at the backend of the manifold 500.

Similarly, the suction areas 508 can have one or more front suction openings 520 at the front side 504 of the manifold 500, which are defined by the suction passages crossing through the manifold 500. The suction tubes 308 pass through the front suction openings 520 and enter the suction passages in the manifold 500. The suction passages also define one or more back suction openings 528 at the backside of the manifold. In certain embodiments, the suction passage has multiple branches at the front end of the manifold 500. Each of the suction tubes 308 enters into a respective branch of the suction passage. The multiple branches converge into a single branch at the backend of the manifold. That single branch defines the back suction opening 528.

FIG. 5(B) illustrates that, in certain embodiments, instead of a single manifold that receives both the delivery tubes 304 and the suction tubes 308, the system can employ a delivery manifold 552 that receives the delivery tubes 304 and a suction manifold 556 that receives the suction tubes 308. Similarly, each manifold 552, 556 can have multiple branches at the frontend and one branch at the backend.

In certain embodiments, the back delivery opening 524 of a manifold is in fluid communication with the delivery module 178 of the backend apparatus 170 via a delivery tubing 572. In certain embodiments, the back suction opening 528 of a manifold is in fluid communication with the suction module 190 via a suction tubing 574.

Referring back to FIG. 1, in certain embodiments, the backend apparatus 170 can have a fluid module 182 that has fluid reservoir containing one or more fluids. In certain embodiments, the fluid module 182 can employ a heat source to heat the fluids in the fluid reservoir to generate the heated fluids. For example, water stored in a water reservoir can be heated to generate steam in the fluid module 182. The fluids, including the heated fluids such as a steam, can be released to the delivery module 178.

In certain embodiments, the backend apparatus 170 can have an agent module 186 that provides agents such as chemical/pharmacological agents and drugs/medicines to the delivery module 178. The agents can be stored in an agent reservoir. In certain embodiments, the agents are in a particle form.

In certain embodiments, the agents are small particles or are encapsulated in small capsules, which can be moved by the momentum of the fluid. For example, nanospheres can be utilized to contain a drug cocktail having at least one of antibiotic, anti-inflammatory, vasodilator or vasoconstrictor, anti-keritin, anti Veg-F, salicylic acid, and other drugs. In certain embodiments, the small capsules such as the nanospheres are configured to time release the medicines after the nanospheres are delivered at the tissue of the target and infiltrate the tissue. In certain embodiments, a first portion of the nanospheres are configured to release the medicines contained after a first period of time from being delivered at the target, and a second portion of the nanospheres are configured to release the medicines contained after a second period of time from being delivered at the target. The nanospheres can include multiple different agents or medicines. For example, a first portion of the nanospheres each include a first medicine, a second portion of the nanospheres each include a second medicine.

The agent and fluid released into the delivery module 178 can be mixed in the delivery module 178. The flow of the fluid can force the agents to flow with the fluid through the delivery tubing/tubes 124, 156 and reach the delivery opening 132 at the probe tip 112. The delivery module 178 can deliver fluids containing at least one of vapor, steam, warm mist humidified air, lipid emulsifier, medicine/drug, gas, solution, liquid gas, etc. into the delivery tubing and tubes 124, 156. The delivery module 178 can employ necessary mechanism as known by one skilled in the art to expel or facilitate the fluid to travel through the delivery tubing and the tubes 124, 156 and exit at delivery opening 132 of the probe tip 112. The delivery module 178 can have a valve that allows the fluid to flow from the delivery module 178 into the delivery tubing 156 and disallow the fluid to flow from the delivery tubing 156 back to the delivery module 178. In certain embodiments, the fluid, and optionally the agent, travel through the delivery tubing/tubes 124, 156 as a result of its own physical characteristics, for example, the increased fluid pressure in the delivery module 178.

The suction module 190 has necessary mechanism including a suction force generator such as a suction pump, as known by one skilled in the art to generate a vacuum in the suction module 190. The vacuum in the suction module 190 generates a suction force, which is transferred through the suction tubing/tubes 128, 160 to the suction opening 136 of the probe tip 112. When the probe tip 112 is placed over the target such as a gland, the suction force can draw the surrounding matters of a predetermined size, such as the obstruction at or within the gland or the agents supplied by the delivery tubes 124, 156 and that are used, into the suction opening 136. The suction force in the suction tubes/tubing 128, 160 will continue to draw the matters into the suction module 190. The suction module 190 can have necessary mechanisms, such as a waste reservoir, that are used to dispose the matters.

The backend apparatus 170 can have a power module 194 that provides power to the delivery, fluid, agent, suction, and control modules.

The control module 174 controls the suction module 190, the delivery module 178, the agent module 186, and the fluid module 182. For example, the control module 174 can instruct the fluid module 182 to release a fluid to the delivery module 178, the agent module 186 to release agents into the delivery module 178, and the delivery module 178 to deliver the mixture of the fluid and agent to delivery opening 132 of the probe tip 112 through the delivery tubing/tubes 124, 156. The control module 174 can also instruct the suction module 190 to operate the vacuum pump and generate a suction force at the suction opening 136 through the suction tubes/tubing 128, 160 of the probe tip 112.

As an example, the above described system can be used to treat belpharitis in the human eye by removing the debris cap and evacuating the meibomian gland. A surfactant along with a warm steam, provided by the handheld device 100, creates substantially high and often about 100% humidity on the surface of the meibomian gland and will dissolve the cap. Additionally, the agitator 140 such as a brush that is incorporated in the device 100 would remove the cap. The slurry can be aspirated using suction.

The above described system can be utilized and applied by an optometrist or ophthalmologist in the office at the slit-lamp (present in all offices). There are around 40,000 optometrists and 8,000 ophthalmologists practicing in the US alone. This can potentially benefit around ⅓ of the human population over 50 years of age.

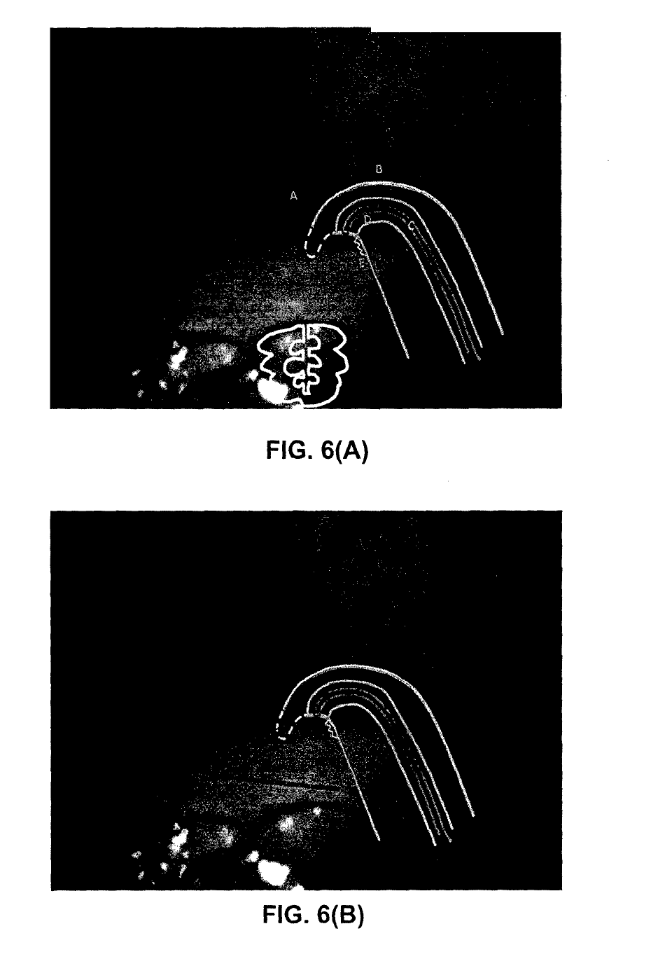

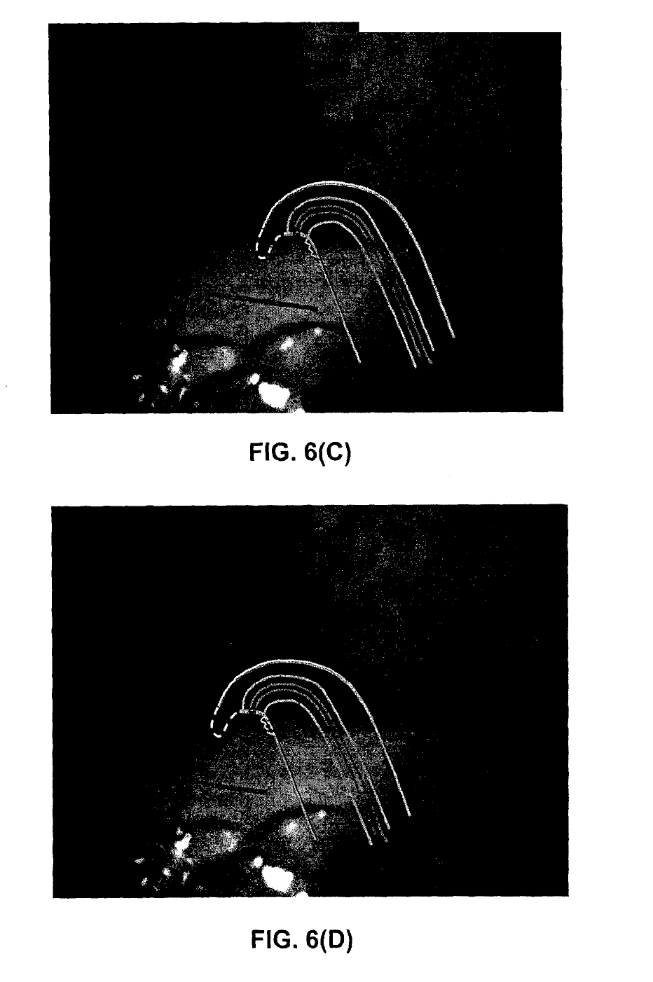

FIG. 6(A) shows an eyelid with blepharitis at 40× magnification. As briefly stated herein above, the upper lid contains approximately 25 meibomian glands and the lower lid contains approximately 20 meibomian glands. Each gland includes a channel into which the secretion flows and an orifice which opens on to the eyelid margin and through which the secretion must flow in order to be added to the tear film upon blinking The glands are of different size, depending upon the location in the eyelid and that the orifice is narrower than the channel.

As briefly mentioned herein above, obstruction composition will vary with the etiology which produced it. However, the obstruction will, in most cases, consist of a combination of, dead cells, keratin, bacteria, desquamated cells, sebaceous ground substance, milky fluid, inspissated or creamy secretions, or any combination of the foregoing in solid, semi-solid and thickened forms. The obstruction may be in the gland channel, at the gland orifice, atop the gland orifice or a combination of the foregoing. As employed herein, obstruction refers to any of the foregoing.

Thus, it is self-evident that any obstruction of the channel will restrict or prevent secretions from exiting the gland and further, that in order to clear such obstructions or “occlusions”, the obstruction may be loosened from the gland wall, and/or broken up, fractured, softened, or liquefied so that it will fit through the gland orifice without causing excessive pain. Lastly, the obstruction remnants must be removed from the gland.

In particular, in FIG. 6(A), the arrows (A) point to the tubular meibomian gland. This gland is occluded with meibum, epithelial cells, and debris from the tears. The outline (B) shows the housing of the probe tip 112. It dips behind the eyelid (dotted lines) for easy movement across the eyelid. The delivery tube has a port which expels a humid mist of heated water and emulsifier (or medication, like doxycycline). The port surrounds the port of delivery tube and is a portion of the suction tube and defines the suction opening. The agitator (E) is placed adjacent to the suction opening. FIG. 6(A) shows a meibomian gland on the lower eyelid. For people skilled in the art, it will be noted that although the detailed description given here relates to the treating the lower eyelid, the process can be equally practiced in relation to the upper eyelid.

FIG. 6(B) shows the progression of a handheld device along the eyelid. The progression may be from nasal to temporal or temporal to nasal or any combination of movement. FIG. 6(C) shows the probe tip positioned directly above a meibomian gland. FIG. 6(D) shows progression of the handheld device toward a next meibomian gland.

For example, when treating a meibomian gland of a patient, optionally a warmed eye mask is positioned over the patient's eyelids to help liquefy or soften the meibomian gland obstructions. Alternative heating mechanism may include conduction, convection and radiation supplied by one or more of the following: thermal conduction, thermal convection, ultrasonic energy, laser energy, RF energy, direct and/or indirect transfer from heat source ad microwave energy which may be applied for a preselected period of time. By varying the amplitude, intensity and length of application, some of the foregoing mechanism may also be employed to fracture or break up the obstruction. In certain embodiments, a closed loop feedback control module, well known to those skilled in the art may be employed during heating to measure temperature proximate the eyelid to ensure that the obstruction does, in fact, reach a temperature sufficient to turn the obstructive material into a flowable, liquid or semi-liquid state. A plate can be then placed behind the eyelid, and a roller tool is gently rolled over the eyelid and eyelid margin to express and unblock the gland.

Further, as show in FIG. 6(A), the practitioner can utilize the curved shape, such as the hook, of the probe tip to open the eyelid and the place probe tip over the gland. The practitioner can operate the control module to the instruct the fluid module and/or the delivery module such that a fluid (such as vapor, steam, warm mist humidified air) are provided from the fluid module to the delivery module. The delivery module delivers the fluid to, or facilitates them to exit at, delivery openings at the probe tip. The fluid can be a heated medium and can include chemical/pharmacological agents. The practitioner place the probe tip over the meibomian gland such that the fluid (e.g. a heated medium) impact the orifice and its surrounding tissues. For example, warm water vapor is delivered with a surfactant, creating 100% humidity on the surface of the eyelid. The heated medium can liquefy, soften, loosen, or fracture the obstruction in the gland as well as dilate the opening of the gland. The surfactant can break apart the obstruction, e.g., lipid plug, at or in the gland. In certain embodiments, the delivery module can deliver vapor with chemical/pharmacological agents to the delivery openings in order to clean the gland margin and removed or exfoliate cells from the gland orifice.

While, or before or after, applying the fluid to the eyelid, the practitioner can use the agitator to apply an external regulated force to the eyelid and/or directly over the obstructed gland to loosen the obstruction within the gland or at the orifice. As discussed above, in certain embodiments, the agitator, such as the standoff pegs, can vibrate at different frequencies. The practitioner can determine the optimum frequency and other parameters such that the agitating force is transmitted through the eyelid tissue to the obstruction. For example, the standoff pegs (or other types of agitators) of the probe tip are in contact with the gland orifice or the tissues surround the orifice. Movement of the standoff pegs can cause the obstruction at or in the gland to be loosened or separated from the gland. The agitation in combination with the application of the fluid, in particular a heated medium, and the surfactant can sufficiently loosen the obstruction in or at the gland as well as dilate the orifice of the gland such that the obstruction can be extracted from the gland without causing much discomfort to the patient.

The control module allows the practitioner to turn on the suction module at any preferred time. The suction module generates a vacuum, which generates a suction force at the suction opening of the probe tip. When the probe tip is placed in close proximity to the gland, the suction force can draw the obstruction at or in the gland into the suction tubes. When the delivery module is operating or has been operated, the suction force can as well evacuate the used fluids, chemical/pharmacological agents, or cellular materials from the gland margin.

In certain embodiments, the agent module can release small agent particles or small capsules containing medication into the delivery module. For example, sticky nanospheres containing a drug cocktail having at least one of antibiotic, anti-inflammatory, vasodilator or vasoconstrictor, anti-keritin, anti Veg-F, salicylic acid or other drugs can be released to the delivery module. The agent module can release a fluid such as warm vapor into the delivery module. In this example, the delivery module delivers the mixture of warm vapor and the drug nanospheres to the probe tip. In certain circumstances, the nanospheres can be considered as being carried by the vapor. The mixture exits the delivery opening of the probe tip and infiltrates the gland. In certain circumstances, the nanospheres can attach to the walls of the gland channel and time release the drug cocktail encapsulated at predetermined time or time intervals. The suction module can be used to aspirate the mixture of the vapor and the drug nanospheres, so that the mixtures does not contact the ocular surface.

In operation, the probe tip will be run along the upper and lower eyelids introducing humidity and other medications such as lipid emulsifiers (Cocamidopropyl Hydroxysultaine, etc), antibiotics (tetracycline, doxycycline, etc), or anything else (anti-inflammatory agents, etc). The suction module will at the same time evacuate the material introduced to the eyelid as well as remove the cap from the meibomian gland and evacuate the contents of the meibomian gland.

The practitioner thus can utilize embodiments of the present disclosure for drug delivery. For example, after cleaning out the meibomian glands, the system can be used to get doxycycline or some other anitibiotic or anti-inflammatory directly into the site.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

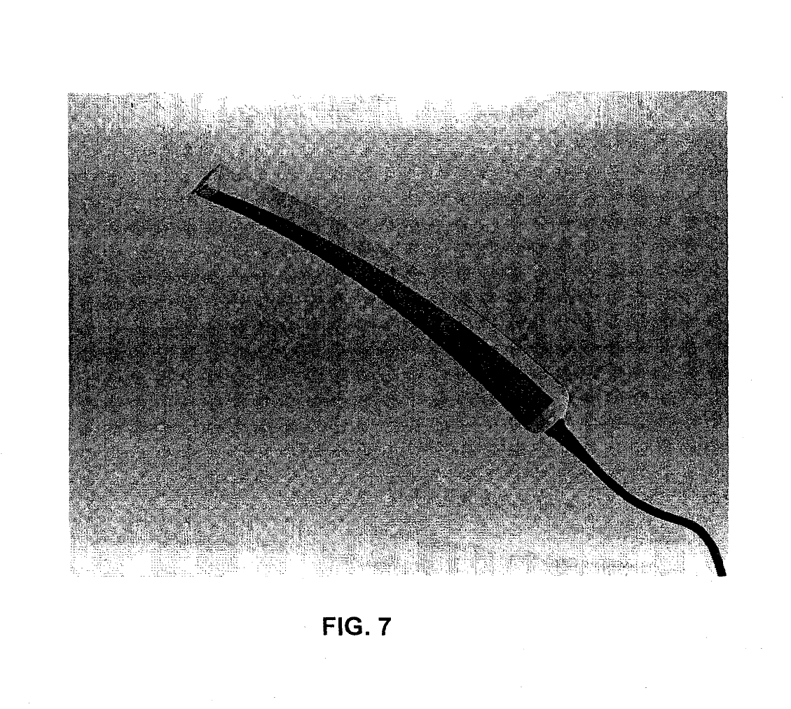

FIG. 7 illustrates another handheld device in accordance with certain embodiments of the present disclosure. The handheld device has an elongate housing that is sized to fit comfortably in a practitioner's hand. For example, the housing can be ergonomically shaped to match contour of the practitioner's fingers and hand, and can rest on a practitioner's hand or wrist. The housing can be coated with textured or rubberized materials to enhance gripping. The housing can have a lid that covers an access opening of the housing. The lid can be opened to allow access to the components or modules placed inside of the housing through the access opening. For example, the lid can be snapped or hinged on the housing. The components or modules inside the housing can be replaced, replaced, or disposed. The housing can contain the delivery tubes and the suction tubes, such as those described above, as well as other necessary mechanical or electrical components. The delivery tubes and the suction tubes can be thermally insulated. For example, the housing can contain a heater, such as the one described above, which can be used to heat the fluid in the delivery tubes. The housing can also contain a agitation or vibration module, such as the one discussed above, that vibrate the handheld device or only the tip of the handheld device. In certain embodiments, the entire handheld device is designed to be a deposable part of the system.

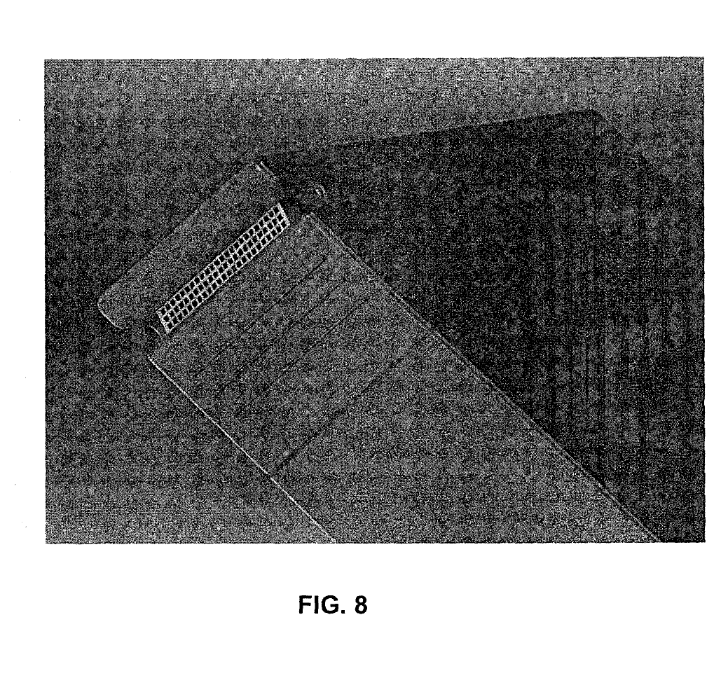

FIG. 8 illustrates the tip of the handheld device in accordance with certain embodiments of the present disclosure. The tip can have a thin, ergonomic shape that promotes precise control. The tip has a treatment side, which has a contact surface and a cartridge recess. A head cartridge, which will be described in more detail below, can be placed, or embedded, in the recess. The head cartridge is coupled with the suction tubes and the delivery tubes, which connected with the suction module or delivery module described above. In the example shown in FIG. 8, the recess is close to the farthest, or top, end of the handheld device. In other words, when a practitioner holds the handheld device, the context service is closer to the practitioner's hand than the cartridge recess. The contact surface can have grooves or a rugged texture. For example, when in use, the practitioner can place the contact surface against skin at the outer side of an eyelid, and then use the friction force generated to move the eyelid away from the eye, thus exposing the tissues and glands at inner side of the eyelid. The recess and the head cartridge are placed at a position above the contact surface such that an exposed target region, such as a meibomian gland, is under the head cartridge for easy treatment. In other words, in certain embodiments, the handheld device does not require using a curved tip, such as a hook, or other similar instruments to deviate or lift the eyelid away from the eye in order to treat the inner side of the eyelid. In certain circumstances, a practitioner can utilize the contact surface to drag the eyelid of a patient and place the head cartridge in the cartridge recess is placed over the target gland or issues on the eyelid. Further, the contact surface, and/or the grooves, can be curved to match the contour of the eyelid.

In certain embodiments, the tip of the handheld device can contain a agitator, such as a brush or scrubber, at, or adjacent to, the contact surface or the cartridge recess. As described above, the agitator can apply an agitating force, such as a vibrating force, to the target or tissues surrounding the target. The agitator force can serve to soften or loosen the target, the surrounding tissues, or the obstruction in a gland.

The head cartridge can be placed in the recess in the removable manner. For example, the head cartridge can be snapped on and off in the recess. Once being properly placed in the recess, the head cartridge is coupled with the delivery tubes and the suction tubes placed in the housing of the handheld device. In certain embodiments, the recess may hold the head cartridge entirely within the recess, such that when the recess is placed over a target region, a space still exist between the target region and the treatment side of the head cartridge.

As will be described in more detail below, the head cartridge can have an encasement that defines delivery, suction, irrigation ports or openings. Those functional ports can vary in size, shape, location and/or configuration. The encasement can be made of soft polymer or other suitable materials as known to one skilled in the art. The encasement can be shaped to match the contour of the eyelid, as well as can apply varying pressure across its width or height.

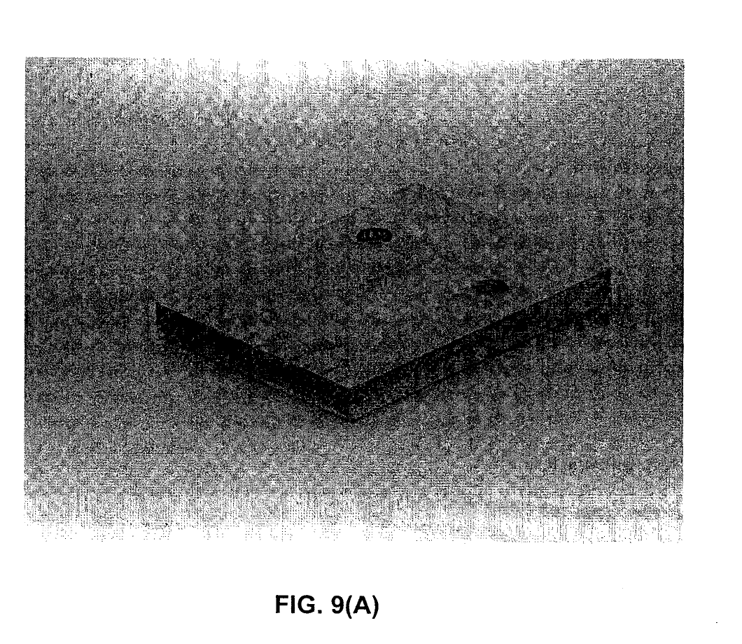

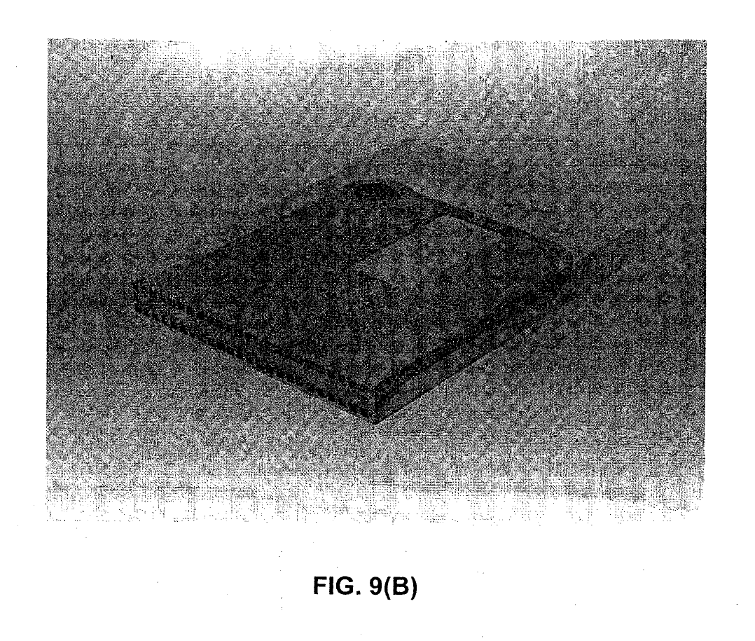

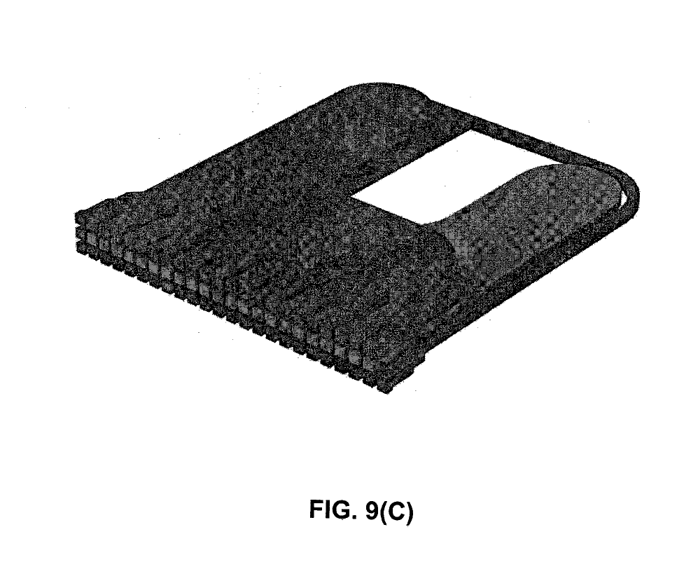

FIG. 9(A)-(C) illustrates a head cartridge in accordance with certain embodiments of the present disclosure. The head cartridge has a suitable shape such that it can be securely placed in the recess of the handheld device. In this specific example shown in FIG. 9(A), the encasement of the head cartridge has a shape of the rectangular parallelepiped. The front side of the encasement defines a treatment surface/side, which defines functional ports or openings such as the delivery, suction, and irrigation ports or openings. Those functional ports can be arranged freely in accordance with specific needs. For example, in the specific example shown in FIG. 9(A), those functional ports are arranged as a matrix.

FIG. 9(C) shows a delivery manifold and a suction manifold that are to be placed in the encasement in accordance with certain embodiments of the present disclosure. The treatment side of the delivery manifold and the suction manifold each have multiple branches that can be placed in the corresponding delivery or suction ports/openings at the treatment side of the encasement. One or more branches at the treatment side can converge into a single branch at a point closer to the backend side. One or more of those branches formed by convergence can further converge into a single branch at a point even closer to the backend side. In other words, at different stages of the manifold from the treatment side to the backend side, the branches in each stage become fewer and fewer. In certain embodiments, eventually, the multiple branches at the treatment side, of either the delivery manifold or the suction manifold, will become a singe branch. The branches at the backend side of the delivery and suction manifold, for example the single branches shown in FIG. 9(C), can be coupled with a delivery tubing/tubes and a suction tubing/tubes (such as the tubing/tubes shown in FIG. 1), respectively. The delivery tubing and the suction tubing are connected with the delivery module and the suction module of the backend apparatus, or alternatively placed in the housing of the handheld device. In certain embodiments, the branches of the suction manifold surround the branches of the delivery manifold.

As a specific example, FIG. 9(A) illustrates the front side of the encasement defines a matrix of openings/ports having 3 rows and 24 columns. Each opening/ports can receive a delivery tube/branch or a suction tube/branch. When placed in the recess of the tip, the columns of the openings/ports align about a longitudinal direction of the handheld device.

FIG. 9(C) illustrates an specific example of the fluid network of the delivery manifold and the suction manifold. The fluid network of the suction manifold, i.e., the suction fluid network, can have 3 layers. The top layer and the bottom layer of the fluid network each have multiple branches, e.g., 24 branches as shown in FIG. 9(c). The middle layer of the suction fluid network has branches between the end branches of the top layer and the bottom layer. The three layers of the fluid network of the suction manifold defines a space in which a layer of the fluid network of the delivery manifold, i.e. the delivery fluid network, is placed. The layer of the delivery fluid network channel has multiple branches, e.g., 22 branches as shown in FIG. 9(c). The branches of the delivery fluid network are surrounded by the branches of the suction fluid network. The three layers of suction fluid network are a common circuit, connected at the rear left main port as shown in FIG. 9(C). The main port can be in fluid communication with the suction tubing/tubing. The delivery fluid network is connected with the delivery tubing/tubes at the rear right main port as shown in FIG. 9(c).

The top and bottom layers of the branches of the suction fluid network can be placed in or can be received by the top and bottom rows of openings/ports of the encasement, respectively. The middle layer of the branches of the suction network can be placed in or can be received by the openings/ports at the both ends of the middle row of the openings/ports of the encasement. The layer of the branches of the delivery fluid network can be placed in or can be received by the middle row of openings/ports, other than the end openings/ports, of the encasement.

The head cartridge with the above configuration can be placed in the recess of the handheld device housing. The handheld device alone or in conjunction with the backend apparatus, as described above, can administer liquid medicine through the delivery openings/ports of head cartridge to a target region (such as a gland) of the patient. The suction openings/ports surrounding the delivery openings/ports can remove the administered liquid medicine as it migrates outward from the target region.

In certain embodiments, the functional modules, such as the control, suction, delivery, fluid, agent, and power modules, contained in the backend apparatus shown in FIG. 1 can be alternatively placed in the housing of the handheld device. A delivery tubing can connect the main port of the delivery manifold with the delivery module. A suction tubing can connect the main port of the suction manifold with the suction module.

The delivery module, regardless where it is located, can have a flow motivation mechanism derived from a diaphragm pump, peristaltic pump, or other type of pump or mechanical means to expel or move the fluid, such as liquid medicine, received from the fluid module, to exit at the head cartridge through the delivery tubing and the delivery manifold.

In certain embodiments, the suction and delivery motivation may be derived from the same pumps/means. In other words, the same motivation mechanism used in the delivery module can as well be used by the suction module to provide a suction force.

Although FIGS. 9(A)-(C) illustrate fluid networks having separate delivery manifold and suction manifold, in certain embodiments a single manifold can implement both the delivery and the suction functions. For example, such a bifunctional manifold can have a single set of branches at the treatment side as well as a delivery main port and a suction main port at the backend side. In use, the bifunctional manifold can be switched to be in fluid communication with the delivery module or the suction module. For example, initially the bifunctional manifold can be in communication with the delivery module but not with the suction module, and the bifunctional manifold is used to administrate a medicine to the target region of the patient. After, the bifunctional manifold is switched to be in fluid communication with the suction module but not with the delivery module, and the suction module can generate a suction force through the bifunctional module and can remove materials at the target region, e.g., the administered medicine.

Further, the waste reservoir may include a rigid container under suction force generator, e.g., a pump or a vacuum. The drug reservoir can include a rigid or flexible container under pressure. The fluid reservoir can contain a medicine or a agent in fluid form. The fluid reservoir can be pressurized by heat/steam generation. The fluid reservoir can be pressurized by mechanical means. A heater can be used to heat the fluid, such as liquid drugs, contained in the fluid reservoir. Parts or all of the functional modules can be disposable. Further, in certain embodiments, the activation of the handheld device may be achieved by button on the device or by a foot pedal connected with the handheld device or the backend apparatus.

### REFERENCES

1. Lemp M A, Nichols K K. Blepharitis in the United States 2009: a survey-based perspective on prevalence and treatment. Ocul Surf. 2009 April; 7(2 Suppl):S1-S14.
2. Nemet A Y, Vinker S, Kaiserman I. Associated morbidity of blepharitis. Ophthalmology. 2011 June; 118(6):1062-8. Epub 2011 Jan. 26.
3. Robb-Nicholson C. By the way, doctor. I suffer from blepharitis and have tried many medications, but the condition always returns. How can I cure it? Hary Womens Health Watch. 2010 September; 18(1):8.

What is claimed is:

1. A system for treating a gland of a patient, comprising:
a handheld device including a probe, a tip of the probe being formed with a suction opening and a delivery opening;
a backend apparatus including
a fluid reservoir configured to contain a fluid,
an agent reservoir configured to contain agent particles, and
a suction force generator;
a delivery channel in fluid communication with the delivery opening and the fluid reservoir and the agent reservoir, the delivery channel allowing a mixture of the fluid and the agent particles to travel through the delivery channel and exit at the delivery opening; and
a suction channel in fluid communication with the suction opening and the suction force generator, the suction channel allowing a suction force being generated at the suction opening by the suction force generator.

2. The system of claim 1, wherein the tip further comprises a shoe adapted to be placed over a gland of a patient, a first side of the shoe having at least one suction area including the suction opening and at least one delivery area including the delivery opening, and the suction area surrounding the delivery area.

3. The system of claim 2, wherein the delivering area has a plurality of discrete areas, and wherein the suction area has a plurality of discrete areas.

4. The system of claim 3, wherein the shoe has a plurality of delivery passages ending at the delivery area, the delivery channel has a plurality of delivery tubes, and each of the delivery tubes is placed in one of the delivery passages; and
wherein the shoe has a plurality of suction passages ending at the suction area, the suction channel has a plurality of suction tubes, and each of the suction tubes is placed in one of the suction passages.

5. The system of claim 2, wherein the shoe has a plurality of protrusions placed adjacent to, and protruding away from, the suction area.

6. The system of claim 5, wherein the plurality of protrusions are standoff pegs configured to separate the gland from the suction opening when the tip is placed over the gland.

7. The system of claim 5, wherein the plurality of protrusions function as an agitator that provides mechanical forces to the gland.

8. The system of claim 7, wherein the gland is a meibomian gland in an eyelid of a patient.

9. The system of claim 8, wherein the plurality of protrusions are configured to vibrate at a predetermined frequency such that the vibration of the protrusions loosens or fractures an obstruction within or at the meibomian gland.

10. The system of claim 1, wherein the handheld device further comprises an agitator at the tip of the probe and configured to generate an agitating force.

11. The system of claim 10, wherein the gland is a meibomian gland in an eyelid of a patient, wherein the agitator is configured to apply a mechanical force to the meibomian gland or tissues of the eyelid adjacent to the meibomian gland, and wherein the mechanical force is sufficient to loosen an obstruction within the meibomian gland.

12. The system of claim 1, wherein the backend apparatus has a delivery module in fluid communication with the fluid reservoir and the agent reservoir, and the delivery module is configured to utilize the fluid to deliver the agent particles to the delivery opening.

13. The system of claim 12, wherein the fluid is a heated fluid.

14. The system of claim 13, wherein the heated fluid is a vapor.

15. The system of claim 1, wherein the backend apparatus has a heating source configured to heat the fluid in the fluid reservoir to generate a heated fluid.

16. The system of claim 1, wherein the gland is a meibomian gland in an eyelid of a patient, and wherein the probe includes a heater at the tip and configured to heat the fluid exiting at the delivery opening to a predetermined temperature such that the heated fluid is sufficient to liquefy or soften an obstruction within or at the meibomian gland.

17. The system of claim 1, wherein the agent particles include nanospheres each containing a medicine.

18. The system of claim 17, wherein a first portion of the nanospheres each include a first medicine, and a second portion of the nanospheres each include a second medicine.

19. The system of claim 17, wherein the nanospheres each include a drug cocktail containing at least one of antibiotic, anti-inflammatory, vasodilator or vasoconstrictor, anti-keritin, anti Veg-F, and salicylic acid.

20. The system of claim 17, wherein the nanospheres are configured to time release the medicines after the nanospheres are delivered at the gland and infiltrate the gland.

21. The system of claim 20, wherein a first portion of the nanospheres are configured to release the medicines contained after a first period of time from being delivered at the gland, and wherein a second portion of the nanospheres are configured to release the medicines contained after a second period of time from being Delivered at the gland.

22. The system of claim 1, wherein the agent particles include particles of a chemical or pharmacological agent.

23. The system of claim 1, wherein the gland is a meibomian gland in an eyelid of a patient, and wherein the tip has a curved shape for ergonomically accessing the meibomian gland.

24. A method for treating a meibomian gland of a patient, comprising:
placing a tip of a probe of a handheld device over a meibomian gland on an eyelid, the tip having a suction opening and a delivery opening; and
operating a backend apparatus, the backend apparatus including
a fluid reservoir having a fluid,
an agent reservoir having agent particles, and
a suction force generator,
such that a delivery channel delivers a mixture of the fluid and the agent particles to exit at the delivery opening and to arrive at the meibomian gland, the delivery channel being in fluid communication with the delivery opening and the fluid reservoir and the agent reservoir; and
such that a suction force is generated at the suction opening by the suction force generator and draws a material at the meibomian gland into a suction channel through the suction opening, the suction channel being in fluid communication with the suction opening and the suction force generator.

25. The method of claim 24, wherein the fluid is a heated at a predetermined temperature.

26. The method of claim 24, wherein the agent particles include nanospheres each containing a medicine.

27. The method of claim 26, wherein a first portion of the nanospheres each include a first medicine, and a second portion of the nanospheres each include a second medicine.

28. The method of claim 26, wherein the nanospheres each include a drug cocktail containing at least one of antibiotic, anti-inflammatory, vasodilator or vasoconstrictor, anti-keritin, anti Veg-F, and salicylic acid.

29. The method of claim 26, wherein the nanospheres are configured to time release the medicines after the nanospheres are delivered at the gland and infiltrate the gland.

30. The method of claim 26, wherein a first portion of the nanospheres are configured to release the medicines contained after a first period of time from being delivered at the gland, and wherein a second portion of the nanospheres are configured to release the medicines contained after a second period of time from being delivered at the gland.

31. The method of claim 24, wherein the agent particles include particles of a chemical or pharmacological agent.

32. The method of claim 24, further comprising operating an agitator of the probe tip to apply a mechanical force at the meibomian gland.

33. An apparatus useable with a handheld device for treating an external tissue of a patient, comprising:
a fluid reservoir configured to contain a fluid;
an agent reservoir configured to contain nanospheres each containing a agent; and
a delivery module configured to deliver a mixture of the fluid and the nanospheres to the handheld device, the delivery module being in fluid communication with the fluid reservoir and the agent reservoir.

34. The apparatus of claim 33, wherein a first portion of the nanospheres each include a first medicine, and a second portion of the nanospheres each include a second medicine.

35. The apparatus of claim 33, wherein the nanospheres each include a drug cocktail containing at least one of antibiotic, anti-inflammatory, vasodilator or vasoconstrictor, anti-keritin, anti Veg-F, and salicylic acid.

36. The apparatus of claim 33, wherein the nanospheres are configured to time release the agents after the nanospheres are delivered at the external tissue and infiltrate the external tissue.

37. The apparatus of claim 33, wherein a first portion of the nanospheres are configured to release the agents contained after a first period of time from being delivered at the external tissue, and wherein a second portion of the nanospheres are configured to release the agents contained after a second period of time from being delivered at the external tissue.

38. The apparatus of claim 33, wherein the external tissue is at a gland of the patient.

39. The apparatus of claim 38, wherein the external tissue is at a meibomian gland in an eyelid of the patient.

40. A handheld device for treating a target region of a patient, comprising:
an elongate housing;
a recess formed at a tip of the elongate housing;
a head cartridge placed in the recess, the head cartridge including a delivery manifold having a plurality of delivery ports at a treatment side of the delivery manifold, the delivery manifold being in fluid communication with a fluid reservoir through a main port of, and at a backend side of, the delivery manifold, the delivery manifold allowing a fluid contained in the fluid reservoir to exit at the delivery ports; and
a contact surface adjacent to the recess, the contact surface having a rugged texture.

41. The handheld device of claim 40, wherein the fluid contained in the fluid reservoir includes a drug cocktail containing at least one of antibiotic, anti-inflammatory, vasodilator or vasoconstrictor, anti-keritin, anti Veg-F, and salicylic acid.

42. The handheld device of claim 40, wherein the fluid contained in the fluid reservoir is a liquid medicine.

43. The handheld device of claim 40, wherein the handheld device further comprises a delivery module that is in fluid communication with the delivery manifold and the fluid reservoir, and wherein the delivery module has a motivation mechanism that expels the fluid to the delivery ports.

44. The handheld device of claim 43, wherein the motivation mechanism includes at least one of a diaphragm pump and a peristaltic pump.

45. The handheld device of claim 40, wherein the handheld device further comprises a suction manifold having a plurality of suction ports at a treatment side of the delivery manifold, the suction manifold being in fluid communication with a suction module through a main port of, and at a backend side of, the suction manifold, the suction manifold allowing the suction module to generate a suction force at the suction ports.

46. The handheld device of claim 40, further comprising an agitator located adjacent to the recess and configured to generate an agitating force.

47. The handheld device of claim 40, further comprising a vibration module configured to vibrate the handheld device.

48. The handheld device of claim 40, further comprising heater placed within the housing, wherein the heater configured to heat the fluid deviled through the delivery manifold.
